(19) United States
(12) Patent Application Publication
(10) Pub. No.: US 2020/0171149 A1
(43) Pub. Date: Jun. 4, 2020

(54) COMBINATION THERAPY FOR NON-SMALL CELL LUNG CANCER POSITIVE FOR EGFR MUTATION

(71) Applicant: MEDIMMUNE LIMITED, CAMBRIDGE (GB)

(72) Inventors: MOHAMMED M. DAR, GAITHERSBURG, MD (US); JOYSON J. KARAKUNNEL, GAITHERSBURG, MD (US); HAIYI JIANG, GAITHERSBURG, MD (US); MAXWELL J. KIRKBY, SHANGHAI (CN)

(21) Appl. No.: 16/788,488

(22) Filed: Feb. 12, 2020

Related U.S. Application Data
(63) Continuation of application No. 15/568,586, filed on Oct. 23, 2017, now abandoned.
(60) Provisional application No. 62/151,739, filed on Apr. 23, 2015.

(51) Int. Cl. A61K 39/395; A61K 45/06; A61K 31/5377; C07K 16/28; A61P 35/00; A61K 9/00

(52) U.S. Cl. CPC A61K 39/3955; A61K 45/06; A61K 31/5377; C07K 16/2827; A61K 2300/00; A61K 9/0019; C07K 2317/71; C07K 2317/76; A61P 35/00

(57) ABSTRACT

The present invention features methods of treating lung cancer (e.g., NSCLC) with an anti-PD-L1 antibody and a tyrosine kinase inhibitor in a subject identified as having an EGFR mutation-positive tumor.

### SEQUENCE LISTING

The instant application contains a Sequence Listing which has been submitted electronically in ASCII format and is hereby incorporated by reference in its entirety. Said ASCII copy, created on Apr. 20, 2016, is named B7IR-200WO1_SL.txt and is 28,134 bytes in size.

### BACKGROUND OF THE INVENTION

Lung cancer is among the most common forms of cancer and is the leading cause of cancer deaths among men and women. More people die of lung cancer annually than of colon, breast, and prostate cancers combined. Non-small cell lung cancer (NSCLC) is the most common form of lung cancer. While the risk of acquiring lung cancer is higher among patients with a history of smoking, lung cancer also affects non-smokers. Improving survival of lung cancer patients remains difficult despite improved medical therapies. Most lung cancer is detected only in advanced stages when therapy options are limited. There is a growing recognition that lung cancer and other malignancies arise from a variety of pathogenic mechanisms. Methods of characterizing these malignancies at a molecular level are useful for stratifying patients, thereby quickly directing them to effective therapies. Improved methods for predicting the responsiveness of subjects having lung cancer, including NSCLC, are urgently required.

### SUMMARY OF THE INVENTION

As described below, the present invention features methods of treating non-small cell lung cancer with an anti-PD-L1 antibody and an Epidermal Growth Factor Receptor (EGFR) tyrosine kinase inhibitor (e.g., gefitinib) in a subject identified as having an EGFR mutation-positive tumor (e.g., deletion in exon 19 of the EGFR gene).

In one aspect, the invention features a method of treating non-small cell lung cancer (NSCLC) in a human patient comprising administering to the patient an anti-PD-L1 antibody, or antigen binding fragment thereof, at a dosage of between about 3 mg/kg and about 10 mg/kg every 2 weeks and an Epidermal Growth Factor Receptor (EGFR) tyrosine kinase inhibitor at about 250 mg per day, thereby treating the NSCLC in the patient.

In another aspect, the invention features a method of treating non-small cell lung cancer (NSCLC) in a human patient comprising administering to the patient an anti-PD-L antibody, or antigen binding fragment thereof, at a dosage of about 3 mg/kg or about 10 mg/kg every 2 weeks and an EGFR tyrosine kinase inhibitor at about 250 mg per day, thereby treating the NSCLC in the patient.

In another aspect, the invention provides a method of treatment involving administering an anti-PD-L1 antibody, or antigen binding fragment thereof, between about 3 mg/kg and about 10 mg/kg every 2 weeks and an EGFR tyrosine kinase inhibitor at about 250 mg per day to a patient identified as having a non-small cell lung cancer that is positive for an EGFR activating mutation.

In another aspect, the invention provides a method of treatment involving administering MEDI4736, or antigen binding fragment thereof, between about 3 mg/kg and about 10 mg/kg every 2 weeks and gefitinib at 250 mg per day to a patient identified as having a non-small cell lung cancer that is positive for an EGFR activating mutation.

In various embodiments of any aspect delineated herein, the anti-PD-L1 antibody has one or more of a heavy chain CDR1 comprising the amino acid scquence GFTSRYWMS (SEQ ID NO: 3); heavy chain CDR2 comprising the amino acid sequence NIKQDGSEKYYVDSVKG (SEQ ID NO: 4); heavy chain CDR3 comprising the amino acid sequence EGGWFGELAFDY (SEQ ID NO: 5); light chain CDR1 comprising the amino acid sequence RASQRVSSSYLA (SEQ ID NO: 6); light chain CDR2 comprising the amino acid sequence DASSRAT (SEQ ID NO: 7); and light chain CDR3 comprising the amino acid sequence QQYGSLPWT (SEQ ID NO: 8). In certain embodiments, the anti-PD-L antibody has one or more of a heavy chain comprising the amino acid sequence:

(SEQ ID NO: 1)
EIVLTQSPGTLSLSPGERATLSCRASQRVSSSYLAWYQQKPGQAPRLLIY
DASSRATGIPDRFSGSGSGTDFTLTISRLEPEDFAVYYCQQYGSLPWTFG
QGTKVEIK

and a light chain comprising the amino acid sequence:

(SEQ ID NO: 2)
EVQLVESGGGLVQPGGSLRLSCAASGFTFSRYWMSWVRQAPGKGLEWVAN
IKQDGSEKYYVDSVKGRFTISRDNAKNSLYLQMNSLRAEDTAVYYCAREG
GWFGELAFDYWGQGTLVTVSS.

In specific embodiments, the anti-PD-L1 antibody is MEDI4736. In various embodiments, the administration of the anti-PD-L1 antibody or MEDI4736, or antigen-binding fragments thereof, is by intravenous infusion.

In various embodiments of any aspect delineated herein, the EGFR tyrosine kinase inhibitor is one or more of gefitinib, erlotinib, icotinib, afatinib, dacomitinib, neratinib, rociletinib, and AZD9291. In various embodiments, the administration of the EGFR tyrosine kinase inhibitor or gefitinib is by oral administration.

In various embodiments of any aspect delineated herein, the anti-PD-L antibody, MEDI4736, or antigen binding fragment thereof, is administered before, during, or after administration of gefitinib. In various embodiments of any aspect delineated herein, the anti-PD-L antibody, MEDI4736, or antigen binding fragment thereof, is administered concurrently with gefitinib.

In various embodiments of any aspect delineated herein, the non-small cell lung cancer is selected from the group consisting of squamous cell carcinoma, adenocarcinoma, large cell carcinoma, adenosquamous carcinoma and sarcomatoid carcinoma.

In various embodiments of any aspect delineated herein, MEDI4736 is administered between about 3 mg/kg to about 10 mg/kg every 2 weeks (e.g., about 3 mg/kg, about 4 mg/kg, about 5 mg/kg, about 6 mg/kg, about 7 mg/kg, about 8 mg/kg, about 9 mg/kg, or about 10 mg/kg every 2 weeks). In various embodiments, MEDI4736 and gefitinib are administered for 8, 10, 12, 16, 20, 24 weeks or more.

In various embodiments of any aspect delineated herein, the treatment stabilizes or decreases one or more of tumor diameter, tumor volume, tumor mass, and tumor burden.

In various embodiments of any aspect delineated herein, EGFR activating mutation is a mutation or deletion in the EGFR kinase domain. In certain embodiments, the deletion encompasses amino acids at positions 746-750 (ELREA) (SEQ ID NO: 13) of an EGFR polypeptide. In specific embodiments, the deletion is in a region encoded by exon 19 of an EGFR nucleic acid molecule. In further embodiments, the EGFR polypeptide comprises a methionine at position 790.

In various embodiments of any aspect delineated herein, the patient is identified as responsive to treatment with an EGFR tyrosine kinase inhibitor. In various embodiments of any aspect delineated herein, the patient is undergoing or has undergone treatment with an EGFR tyrosine kinase inhibitor or gefitinib. In various embodiments of any aspect delineated herein, the treatment increases overall survival as compared to the administration of either EGFR tyrosine kinase inhibitor or gefitinib alone.

Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “Programmed death-ligand 1 (PD-L1) polypeptide” is meant a polypeptide or fragment thereof having at least about 85% amino acid identity to NCBI Accession No. NP_001254635 and having PD-1 and CD80 binding activity. An exemplary PD-L1 amino acid sequence is provided below.

| (SEQ ID NO: 9) | |
|---|---|
| 1 | mrifavfifm tywhllnapy nkinqrilvv dpvtsehelt cqaegypkae viwtssdhqv |
| 61 | lsgkttttns kreeklfnvt stirintttn eifyctfrrl dpeenhtael vipelplahp |
| 121 | pnerthlvil gaillclgva ltfifrlrkg rmmdvkkcgi qdtnskkqsd thleet |

By “PD-L1 nucleic acid molecule” is meant a polynucleotide encoding a PD-L1 polypeptide. An exemplary PD-L1 nucleic acid molecule sequence is provided at NCBI Accession No. NM_001267706.

| (SEQ ID NO: 10) | |
|---|---|
| 1 | ggcgcaacgc tgagcagctg gcgcgtcccg cgcggcccca gttctgcgca gcttcccgag |
| 61 | gctccgcacc agccgcgctt ctgtccgcct gcagggcatt ccagaaagat gaggatattt |
| 121 | gctgtcttta tattcatgac ctactggcat ttgctgaacg ccccatacaa caaaatcaac |
| 181 | caaagaattt tggttgtgga tccagtcacc tctgaacatg aactgacatg tcaggctgag |
| 241 | ggctacccca aggccgaagt catctggaca agcagtgacc atcaagtcct gagtggtaag |
| 301 | accaccacca ccaattccaa gagagaggag aagcttttca atgtgaccag cacactgaga |
| 361 | atcaacacaa caactaatga gattttctac tgcactttta ggagattaga tcctgaggaa |
| 421 | aaccatacag ctgaattggt catcccagaa ctacctctgg cacatcctcc aaatgaaagg |
| 481 | actcacttgg taattctggg agccatctta ttatgccttg gtgtagcact gacattcatc |
| 541 | ttccgtttaa gaaaagggag aatgatggat gtgaaaaaat gtggcatcca agatacaaac |
| 601 | tcaaagaagc aaagtgatac acatttggag gagacgtaat ccagcattgg aacttctgat |
| 661 | cttcaagcag ggattctcaa cctgtggttt aggggttcat cggggctgag cgtgacaaga |
| 721 | ggaaggaatg ggcccgtggg atgcaggcaa tgtgggactt aaaaggccca agcactgaaa |
| 781 | atggaacctg gcgaaagcag aggaggagaa tgaagaaaga tggagtcaaa cagggagcct |
| 841 | ggagggagac cttgatactt tcaaatgcct gaggggctca tcgacgcctg tgacagggag |
| 901 | aaaggatact tctgaacaag gagcctccaa gcaaatcatc cattgctcat cctaggaaga |
| 961 | cgggttgaga atccctaatt tgagggtcag ttcctgcaga agtgcccttt gcctccactc |
| 1021 | aatgcctcaa tttgttttct gcatgactga gagtctcagt gttggaacgg gacagtattt |
| 1081 | atgtatgagt ttttcctatt tattttgagt ctgtgaggtc ttcttgtcat gtgagtgtgg |
| 1141 | ttgtgaatga tttcttttga agatatattg tagtagatgt tacaattttg tcgccaaact |
| 1201 | aaacttgctg cttaatgatt tgctcacatc tagtaaaaca tggagtattt gtaaggtgct |
| 1261 | tggtctcctc tataactaca agtatacatt ggaagcataa agatcaaacc gttggttgca |
| 1321 | taggatgtca cctttattta acccattaat actctggttg acctaatctt attctcagac |
| 1381 | ctcaagtgtc tgtgcagtat ctgttccatt taaatatcag ctttacaatt atgtggtagc |
| 1441 | ctacacacat aatctcattt catcgctgta accaccctgt tgtgataacc actattattt |
| 1501 | tacccatcgt acagctgagg aagcaaacag attaagtaac ttgcccaaac cagtaaatag |
| 1561 | cagacctcag actgccaccc actgtccttt tataatacaa tttacagcta tattttactt |
| 1621 | taagcaattc ttttattcaa aaaccattta ttaagtgccc ttgcaatatc aatcgctgtg |
| 1681 | ccaggcattg aatctacaga tgtgagcaag acaaagtacc tgtcctcaag gagctcatag |
| 1741 | tataatgagg agattaacaa gaaaatgtat tattacaatt tagtccagtg tcatagcata |
| 1801 | aggatgatgc gaggggaaaa cccgagcagt gttgccaaga ggaggaaata ggccaatgtg |
| 1861 | gtctgggacg gttggatata cttaaacatc ttaataatca gagtaatttt catttacaaa |
| 1921 | gagaggtcgg tacttaaaat aaccctgaaa aataacactg gaattccttt tctagcatta |
| 1981 | tatttattcc tgatttgcct ttgccatata atctaatgct tgtttatata gtgtctggta |
| 2041 | ttgtttaaca gttctgtctt ttctatttaa atgccactaa attttaaatt catacctttc |
| 2101 | catgattcaa aattcaaaag atcccatggg agatggttgg aaaatctcca cttcatcctc |
| 2161 | caagccattc aagtttcctt tccagaagca actgctactg cctttcattc atatgttctt |
| 2221 | ctaaagatag tctacatttg gaaatgtatg ttaaaagcac gtatttttaa aatttttttc |
| 2281 | ctaaatagta acacattgta tgtctgctgt gtactttgct atttttattt attttagtgt |
| 2341 | ttcttatata gcagatggaa tgaatttgaa gttcccaggg ctgaggatcc atgccttctt |
| 2401 | tgtttctaag ttatctttcc catagctttt cattatcttt catatgatcc agtatatgtt |
| 2461 | aaatatgtcc tacatataca tttagacaac caccatttgt taagtatttg ctctaggaca |
| 2521 | gagtttggat ttgtttatgt ttgctcaaaa ggagacccat gggctctcca gggtgcactg |
| 2581 | agtcaatcta gtcctaaaaa gcaatcttat tattaactct gtatgacaga atcatgtctg |
| 2641 | gaacttttgt tttctgcttt ctgtcaagta taaacttcac tttgatgctg tacttgcaaa |
| 2701 | atcacatttt ctttctggaa attccggcag tgtaccttga ctgctagcta ccctgtgcca |
| 2761 | gaaaagcctc attcgttgtg cttgaaccct tgaatgccac cagctgtcat cactacacag |
| 2821 | ccctcctaag aggcttcctg gaggtttcga gattcagatg ccctgggaga tcccagagtt |
| 2881 | tcctttccct cttggccata ttctggtgtc aatgacaagg agtaccttgg ctttgccaca |
| 2941 | tgtcaaggct gaagaaacag tgtctccaac agagctcctt gtgttatctg tttgtacatg |
| 3001 | tgcatttgta cagtaattgg tgtgacagtg ttctttgtgt gaattacagg caagaattgt |
| 3061 | ggctgagcaa ggcacatagt ctactcagtc tattcctaag tcctaactcc tccttgtggt |
| 3121 | gttggatttg taaggcactt tatccctttt gtctcatgtt tcatcgtaaa tggcataggc |
| 3181 | agagatgata cctaattctg catttgattg tcactttttg tacctgcatt aatttaataa |
| 3241 | aatattctta tttattttgt tacttggtac accagcatgt ccattttctt gtttattttg |
| 3301 | tgtttaataa aatgttcagt ttaacatccc agtggagaaa gttaaaaaa |

By “anti-PD-L antibody” is meant an antibody that selectively binds a PD-L polypeptide. Exemplary anti-PD-L antibodies are described for example at U.S. Pat. No. 8,779,108/U.S. Publ. No. 20130034559, the disclosures of which are incorporated herein by reference in their entirety. In one particular embodiment, the anti-PD-L antibody is MEDI4736, which has the following CDR and heavy and light chain sequences:

MEDI4736 VH CDR1
(SEQ ID NO: 3)
GFTFSRYWMS
MEDI4736 VH CDR2
(SEQ ID NO: 4)
NIKQDGSEKYYVDSVKG
MEDI4736 VH CDR3
(SEQ ID NO: 5)
EGGWFGELAFDY
MEDI4736 VL CDR1
(SEQ ID NO: 6)
RASQRVSSSYLA
MEDI4736 VL CDR2
(SEQ ID NO: 7)
DASSRAT
MEDI4736 VL CDR3
(SEQ ID NO: 8)
QQYGSLPWT
MEDI4736 Heavy chain
(SEQ ID NO: 2)
EVQLVESGGGLVQPGGSLRLSCAASGFTFSRYWMSWVRQAPGKGLEWVAN
IKQDGSEKYYVDSVKGRFTISRDNAKNSLYLQMNSLRAEDTAVYYCAREG
GWFGELAFDYWGQGTLVTVSS
MEDI4736 Light chain
(SEQ ID NO: 1)
EIVLTQSPGTLSLSPGERATLSCRASQRVSSSYLAWYQQKPGQAPRLLIY
DASSRATGIPDRFSGSGSGTDFTLTISRLEPEDFAVYYCQQYGSLPWTFG
QGTKVEIK

By “Epidermal growth factor receptor (EGFR) polypeptide” is meant a polypeptide or fragment thereof having at least about 85% amino acid identity to NCBI Accession No. NP_005219 and having tyrosine kinase activity. An exemplary EGFR amino acid sequence is provided below.

| (SEQ ID NO: 11) | |
|---|---|
| 1 | mrpsgtagaa llallaalcp asraleekkv cqgtsnkltq lgtfedhfls lqrmfnncev |
| 61 | vlgnleityv qrnydlsflk tiqevagyvl ialntverip lenlqiirgn myyensyala |
| 121 | vlsnydankt glkelpmrnl qeilhgavrf snnpalcnve siqwrdivss dflsnmsmdf |
| 181 | qnhlgscqkc dpscpngscw gageencqkl tkiicaqqcs grcrgkspsd cchnqcaagc |
| 241 | tgpresdclv crkfrdeatc kdtcpplmly npttyqmdvn pegkysfgat cvkkcprnyv |
| 301 | vtdhgscvra cgadsyemee dgvrkckkce gpcrkvcngi gigefkdsls inatnikhfk |
| 361 | nctsisgdlh ilpvafrgds fthtppldpq eldilktvke itgflliqaw penrtdlhaf |
| 421 | enleiirgrt kqhgqfslav vslnitslgl rslkeisdgd viisgnknlc yantinwkkl |
| 481 | fgtsgqktki isnrgensck atgqvchalc spegcwgpep rdcvscrnvs rgrecvdkcn |
| 541 | llegeprefv enseciqchp eclpqamnit ctgrgpdnci qcahyidgph cvktcpagvm |
| 601 | genntlvwky adaghvchlc hpnctygctg pglegcptng pkipsiatgm vgalllllvv |
| 661 | algiglfmrr rhivrkrtlr rllqerelve pltpsgeapn qallrilket efkkikvlgs |
| 721 | gafgtvykgl wipegekvki pvaikelrea tspkankeil deayvmasvd nphvcrllgi |
| 781 | cltstvqlit qlmpfgclld yvrehkdnig sqyllnwcvq iakgmnyled rrlvhrdlaa |
| 841 | rnvlvktpqh vkitdfglak llgaeekeyh aeggkvpikw malesilhri ythqsdvwsy |
| 901 | gvtvwelmtf gskpydgipa seissilekg erlpqppict idvymimvkc wmidadsrpk |
| 961 | freliiefsk mardpqrylv iqgdermhlp sptdsnfyra lmdeedmddv vdadeylipq |
| 1021 | qgffsspsts rtpllsslsa tsnnstvaci drnglqscpi kedsflqrys sdptgalted |
| 1081 | siddtflpvp eyinqsvpkr pagsvqnpvy hnqpinpaps rdphyqdphs tavgnpeyln |
| 1141 | tvqptcvnst fdspahwaqk gshqisldnp dyqqdffpke akpngifkgs taenaeylrv |
| 1201 | apqssefiga |

In various embodiments, the EGFR contains an activating mutation. In some embodiments, EGFR containing mutations are more sensitive to tyrosine kinase inhibitors compared to wild-type EGFR. In certain embodiments, the EGFR contains a deletion comprising the amino acids at positions 746-750 (ELREA) (SEQ ID NO: 13).

By “EGFR nucleic acid molecule” is meant a polynucleotide encoding an EGFR polypeptide. An exemplary EGFR nucleic acid molecule sequence is provided at NCBI Accession No. NM_005228, which is reproduced below:

| (SEQ ID NO: 12) | |
|---|---|
| 1 | ccccggcgca gcgcggccgc agcagcctcc gccccccgca cggtgtgagc gcccgacgcg |
| 61 | gccgaggcgg ccggagtccc gagctagccc cggcggccgc cgccgcccag accggacgac |
| 121 | aggccacctc gtcggcgtcc gcccgagtcc ccgcctcgcc gccaacgcca caaccaccgc |
| 181 | gcacggcccc ctgactccgt ccagtattga tcgggagagc cggagcgagc tcttcgggga |
| 241 | gcagcgatgc gaccctccgg gacggccggg gcagcgctcc tggcgctgct ggctgcgctc |
| 301 | tgcccggcga gtcgggctct ggaggaaaag aaagtttgcc aaggcacgag taacaagctc |
| 361 | acgcagttgg gcacttttga agatcatttt ctcagcctcc agaggatgtt caataactgt |
| 421 | gaggtggtcc ttgggaattt ggaaattacc tatgtgcaga ggaattatga tctttccttc |
| 481 | ttaaagacca tccaggaggt ggctggttat gtcctcattg ccctcaacac agtggagcga |
| 541 | attcctttgg aaaacctgca gatcatcaga ggaaatatgt actacgaaaa ttcctatgcc |
| 601 | ttagcagtct tatctaacta tgatgcaaat aaaaccggac tgaaggagct gcccatgaga |
| 661 | aatttacagg aaatcctgca tggcgccgtg cggttcagca acaaccctgc cctgtgcaac |
| 721 | gtggagagca tccagtggcg ggacatagtc agcagtgact ttctcagcaa catgtcgatg |
| 781 | gacttccaga accacctggg cagctgccaa aagtgtgatc caagctgtcc caatgggagc |
| 841 | tgctggggtg caggagagga gaactgccag aaactgacca aaatcatctg tgcccagcag |
| 901 | tgctccgggc gctgccgtgg caagtccccc agtgactgct gccacaacca gtgtgctgca |
| 961 | ggctgcacag gcccccggga gagcgactgc ctggtctgcc gcaaattccg agacgaagcc |
| 1021 | acgtgcaagg acacctgccc cccactcatg ctctacaacc ccaccacgta ccagatggat |
| 1081 | gtgaaccccg agggcaaata cagctttggt gccacctgcg tgaagaagtg tccccgtaat |
| 1141 | tatgtggtga cagatcacgg ctcgtgcgtc cgagcctgtg gggccgacag ctatgagatg |
| 1201 | gaggaagacg gcgtccgcaa gtgtaagaag tgcgaagggc cttgccgcaa agtgtgtaac |
| 1261 | ggaataggta ttggtgaatt taaagactca ctctccataa atgctacgaa tattaaacac |
| 1321 | ttcaaaaact gcacctccat cagtggcgat ctccacatcc tgccggtggc atttaggggt |
| 1381 | gactccttca cacatactcc tcctctggat ccacaggaac tggatattct gaaaaccgta |
| 1441 | aaggaaatca cagggttttt gctgattcag gcttggcctg aaaacaggac ggacctccat |
| 1501 | gcctttgaga acctagaaat catacgcggc aggaccaagc aacatggtca gttttctctt |
| 1561 | gcagtcgtca gcctgaacat aacatccttg ggattacgct ccctcaagga gataagtgat |
| 1621 | ggagatgtga taatttcagg aaacaaaaat ttgtgctatg caaatacaat aaactggaaa |
| 1681 | aaactgtttg ggacctccgg tcagaaaacc aaaattataa gcaacagagg tgaaaacagc |
| 1741 | tgcaaggcca caggccaggt ctgccatgcc ttgtgctccc ccgagggctg ctggggcccg |
| 1801 | gagcccaggg actgcgtctc ttgccggaat gtcagccgag gcagggaatg cgtggacaag |
| 1861 | tgcaaccttc tggagggtga gccaagggag tttgtggaga actctgagtg catacagtgc |
| 1921 | cacccagagt gcctgcctca ggccatgaac atcacctgca caggacgggg accagacaac |
| 1981 | tgtatccagt gtgcccacta cattgacggc ccccactgcg tcaagacctg cccggcagga |
| 2041 | gtcatgggag aaaacaacac cctggtctgg aagtacgcag acgccggcca tgtgtgccac |
| 2101 | ctgtgccatc caaactgcac ctacggatgc actgggccag gtcttgaagg ctgtccaacg |
| 2161 | aatgggccta agatcccgtc catcgccact gggatggtgg gggccctcct cttgctgctg |
| 2221 | gtggtggccc tggggatcgg cctcttcatg cgaaggcgcc acatcgttcg gaagcgcacg |
| 2281 | ctgcggaggc tgctgcagga gagggagctt gtggagcctc ttacacccag tggagaagct |
| 2341 | cccaaccaag ctctcttgag gatcttgaag gaaactgaat tcaaaaagat caaagtgctg |
| 2401 | ggctccggtg cgttcggcac ggtgtataag ggactctgga tcccagaagg tgagaaagtt |
| 2461 | aaaattcccg tcgctatcaa ggaattaaga gaagcaacat ctccgaaagc caacaaggaa |
| 2521 | atcctcgatg aagcctacgt gatggccagc gtggacaacc cccacgtgtg ccgcctgctg |
| 2581 | ggcatctgcc tcacctccac cgtgcagctc atcacgcagc tcatgccctt cggctgcctc |
| 2641 | ctggactatg tccgggaaca caaagacaat attggctccc agtacctgct caactggtgt |
| 2701 | gtgcagatcg caaagggcat gaactacttg gaggaccgtc gcttggtgca ccgcgacctg |
| 2761 | gcagccagga acgtactggt gaaaacaccg cagcatgtca agatcacaga ttttgggctg |
| 2821 | gccaaactgc tgggtgcgga agagaaagaa taccatgcag aaggaggcaa agtgcctatc |
| 2881 | aagtggatgg cattggaatc aattttacac agaatctata cccaccagag tgatgtctgg |
| 2941 | agctacgggg tgaccgtttg ggagttgatg acctttggat ccaagccata tgacggaatc |
| 3001 | cctgccagcg agatctcctc catcctggag aaaggagaac gcctccctca gccacccata |
| 3061 | tgtaccatcg atgtctacat gatcatggtc aagtgctgga tgatagacgc agatagtcgc |
| 3121 | ccaaagttcc gtgagttgat catcgaattc tccaaaatgg cccgagaccc ccagcgctac |
| 3181 | cttgtcattc agggggatga aagaatgcat ttgccaagtc ctacagactc caacttctac |
| 3241 | cgtgccctga tggatgaaga agacatggac gacgtggtgg atgccgacga gtacctcatc |
| 3301 | ccacagcagg gcttcttcag cagcccctcc acgtcacgga ctcccctcct gagctctctg |
| 3361 | agtgcaacca gcaacaattc caccgtggct tgcattgata gaaatgggct gcaaagctgt |
| 3421 | cccatcaagg aagacagctt cttgcagcga tacagctcag accccacagg cgccttgact |
| 3481 | gaggacagca tagacgacac cttcctccca gtgcctgaat acataaacca gtccgttccc |
| 3541 | aaaaggcccg ctggctctgt gcagaatcct gtctatcaca atcagcctct gaaccccgcg |
| 3601 | cccagcagag acccacacta ccaggacccc cacagcactg cagtgggcaa ccccgagtat |
| 3661 | ctcaacactg tccagcccac ctgtgtcaac agcacattcg acagccctgc ccactgggcc |
| 3721 | cagaaaggca gccaccaaat tagcctggac aaccctgact accagcagga cttctttccc |
| 3781 | aaggaagcca agccaaatgg catctttaag ggctccacag ctgaaaatgc agaataccta |
| 3841 | agggtcgcgc cacaaagcag tgaatttatt ggagcatgac cacggaggat agtatgagcc |
| 3901 | ctaaaaatcc agactctttc gatacccagg accaagccac agcaggtcct ccatcccaac |
| 3961 | agccatgccc gcattagctc ttagacccac agactggttt tgcaacgttt acaccgacta |
| 4021 | gccaggaagt acttccacct cgggcacatt ttgggaagtt gcattccttt gtcttcaaac |
| 4081 | tgtgaagcat ttacagaaac gcatccagca agaatattgt ccctttgagc agaaatttat |
| 4141 | ctttcaaaga ggtatatttg aaaaaaaaaa aaagtatatg tgaggatttt tattgattgg |
| 4201 | ggatcttgga gtttttcatt gtcgctattg atttttactt caatgggctc ttccaacaag |
| 4261 | gaagaagctt gctggtagca cttgctaccc tgagttcatc caggcccaac tgtgagcaag |
| 4321 | gagcacaagc cacaagtctt ccagaggatg cttgattcca gtggttctgc ttcaaggctt |
| 4381 | ccactgcaaa acactaaaga tccaagaagg ccttcatggc cccagcaggc cggatcggta |
| 4441 | ctgtatcaag tcatggcagg tacagtagga taagccactc tgtcccttcc tgggcaaaga |
| 4501 | agaaacggag gggatggaat tcttccttag acttactttt gtaaaaatgt ccccacggta |
| 4561 | cttactcccc actgatggac cagtggtttc cagtcatgag cgttagactg acttgtttgt |
| 4621 | cttccattcc attgttttga aactcagtat gctgcccctg tcttgctgtc atgaaatcag |
| 4681 | caagagagga tgacacatca aataataact cggattccag cccacattgg attcatcagc |
| 4741 | atttggacca atagcccaca gctgagaatg tggaatacct aaggatagca ccgcttttgt |
| 4801 | tctcgcaaaa acgtatctcc taatttgagg ctcagatgaa atgcatcagg tcctttgggg |
| 4861 | catagatcag aagactacaa aaatgaagct gctctgaaat ctcctttagc catcacccca |
| 4921 | accccccaaa attagtttgt gttacttatg gaagatagtt ttctcctttt acttcacttc |
| 4981 | aaaagctttt tactcaaaga gtatatgttc cctccaggtc agctgccccc aaaccccctc |
| 5041 | cttacgcttt gtcacacaaa aagtgtctct gccttgagtc atctattcaa gcacttacag |
| 5101 | ctctggccac aacagggcat tttacaggtg cgaatgacag tagcattatg agtagtgtgg |
| 5161 | aattcaggta gtaaatatga aactagggtt tgaaattgat aatgctttca caacatttgc |
| 5221 | agatgtttta gaaggaaaaa agttccttcc taaaataatt tctctacaat tggaagattg |
| 5281 | gaagattcag ctagttagga gcccaccttt tttcctaatc tgtgtgtgcc ctgtaacctg |
| 5341 | actggttaac agcagtcctt tgtaaacagt gttttaaact ctcctagtca atatccaccc |
| 5401 | catccaattt atcaaggaag aaatggttca gaaaatattt tcagcctaca gttatgttca |
| 5461 | gtcacacaca catacaaaat gttccttttg cttttaaagt aatttttgac tcccagatca |
| 5521 | gtcagagccc ctacagcatt gttaagaaag tatttgattt ttgtctcaat gaaaataaaa |
| 5581 | ctatattcat ttccactcta aaaaaaaaaa aaaaaa |

In various embodiments, the EGFR nucleic acid molecule contains an in-frame deletion in exon 19, which encodes part of the EGFR kinase domain.

By “Tyrosine Kinase Inhibitor (TKI) molecule” is meant an inhibitor of the tyrosine kinase domain of a receptor tyrosine kinase (e.g., EGFR). In some embodiments, a tyrosine kinase inhibitor specifically binds and/or inhibits the kinase activity of a specific receptor tyrosine kinase domain. Thus, TKIs can discriminate between protein tyrosine kinases that are closely related by sequence identity.

By “EGFR Tyrosine Kinase Inhibitor (TKI) molecule” is meant a compound that specifically binds the kinase domain and/or inhibits the kinase activity of an EGFR polypeptide. In various embodiments, EGFR tyrosine kinase inhibitors include gefitinib, erlotinib, afatinib, and AZD9291. In particular embodiments, subjects identified as having EGFR mutation positive non-small cell lung cancer are selected for treatment with an EGFR tyrosine kinase inhibitor and an anti-PD-L1 antibody.

By “(Gefitinib” is meant the compound having the following formula:

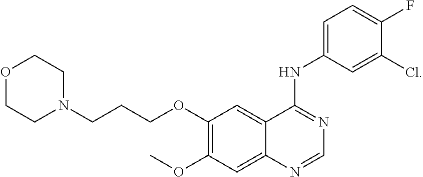 [SMILES: COc1cc2ncnc(Nc3ccc(F)c(Cl)c3)c2cc1OCCCN1CCOCC1]

Gefitinib (CAS no. 184475-35-2) is also known as N-(3-chloro-4-fluorophenyl)-7-methoxy-6-(3-morpholinopropoxy)quinazolin-4-amine, N-(3-chloro-4-fluorophenyl)-7-methoxy-6-[3-(4-morpholinyl)propoxy)]quinazolin-4-amine, and by the trade name IRESSA® (AstraZeneca). Gefitinib is described for example at U.S. Pat. No. 5,770,599, the disclosure of which is incorporated herein by reference in its entirety.

The term “antibody,” as used in this disclosure, refers to an immunoglobulin or a fragment or a derivative thereof, and encompasses any polypeptide comprising an antigen-binding site, regardless whether it is produced in vitro or in vivo. The term includes, but is not limited to, polyclonal, monoclonal, monospecific, polyspecific, non-specific, humanized, single-chain, chimeric, synthetic, recombinant, hybrid, mutated, and grafted antibodies. Unless otherwise modified by the term “intact,” as in “intact antibodies,” for the purposes of this disclosure, the term “antibody” also includes antibody fragments such as Fab, F(ab′)₂, Fv, scFv, Fd, dAb, and other antibody fragments that retain antigen-binding function, i.e., the ability to bind PD-L1 specifically. Typically, such fragments would comprise an antigen-binding domain.

The terms “antigen-binding domain,” “antigen-binding fragment,” and “binding fragment” refer to a part of an antibody molecule that comprises amino acids responsible for the specific binding between the antibody and the antigen. In instances, where an antigen is large, the antigen-binding domain may only bind to a part of the antigen. A portion of the antigen molecule that is responsible for specific interactions with the antigen-binding domain is referred to as “epitope” or “antigenic determinant.” An antigen-binding domain typically comprises an antibody light chain variable region (V_{L}) and an antibody heavy chain variable region (Vii), however, it does not necessarily have to comprise both. For example, a so-called Fd antibody fragment consists only of a VH domain, but still retains some antigen-binding function of the intact antibody.

Binding fragments of an antibody are produced by recombinant DNA techniques, or by enzymatic or chemical cleavage of intact antibodies. Binding fragments include Fab, Fab′, F(ab′)2, Fv, and single-chain antibodies. An antibody other than a “bispecific” or “bifunctional” antibody is understood to have each of its binding sites identical. Digestion of antibodies with the enzyme, papain, results in two identical antigen-binding fragments, known also as “Fab” fragments, and a “Fc” fragment, having no antigen-binding activity but having the ability to crystallize. Digestion of antibodies with the enzyme, pepsin, results in the a F(ab′)2 fragment in which the two arms of the antibody molecule remain linked and comprise two-antigen binding sites. The F(ab′)2 fragment has the ability to crosslink antigen. “Fv” when used herein refers to the minimum fragment of an antibody that retains both antigen-recognition and antigen-binding sites. “Fab” when used herein refers to a fragment of an antibody that comprises the constant domain of the light chain and the CHI domain of the heavy chain.

The term “mAb” refers to monoclonal antibody. Antibodies of the invention comprise without limitation whole native antibodies, bispecific antibodies; chimeric antibodies; Fab, Fab′, single chain V region fragments (scFv), fusion polypeptides, and unconventional antibodies.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “reference” is meant a standard of comparison.

By “responsive” in the context of therapy is meant susceptible to treatment.

By “specifically binds” is meant a compound (e.g., antibody) that recognizes and binds a molecule (e.g., polypeptide), but which does not substantially recognize and bind other molecules in a sample, for example, a biological sample. For example, two molecules that specifically bind form a complex that is relatively stable under physiologic conditions. Specific binding is characterized by a high affinity and a low to moderate capacity as distinguished from nonspecific binding which usually has a low affinity with a moderate to high capacity. Typically, binding is considered specific when the affinity constant K_{A} is higher than 10⁶ M⁻¹, or more preferably higher than 10⁸ M⁻¹. If necessary, non-specific binding can be reduced without substantially affecting specific binding by varying the binding conditions. The appropriate binding conditions such as concentration of antibodies, ionic strength of the solution, temperature, time allowed for binding, concentration of a blocking agent (e.g., serum albumin, milk casein), etc., may be optimized by a skilled artisan using routine techniques.

By “subject” is meant a mammal, including, but not limited to, a human or non-human mammal, such as a bovine, equine, canine, ovine, or feline. In particular embodiments, the subject is a human patient having non-small cell lung cancer (NSCLC). There are three main subtypes of NSCLC: squamous cell carcinoma, adenocarcinoma, and large cell (undifferentiated) carcinoma. Other subtypes include adenosquamous carcinoma and sarcomatoid carcinoma.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### DETAILED DESCRIPTION OF THE INVENTION

As described below, the present invention features methods of treating non-small cell lung cancer with an anti-PD-L1 antibody and an EGFR tyrosine kinase inhibitor (e.g., gefitinib) in a subject (e.g., a subject identified as having a non-small cell lung cancer tumor positive for an EGFR activating mutation).

### Programmed Death-Ligand 1 (PD-L1)

The role of the immune system, in particular T cell-mediated cytotoxicity, in tumor control is well recognized. Although control of tumor growth and survival by T cells in cancer patients in early and late stages of the disease have been shown, tumor-specific T-cell responses are difficult to mount and sustain in cancer patients.

One T cell modulatory pathway receiving significant attention to date signals through programmed death ligand 1 (PD-L1, also known as B7H-1 or CD274). PD-L1 is also part of a complex system of receptors and ligands that are involved in controlling T cell activation. In normal tissue, PD-L1 is expressed on T cells, B cells, dendritic cells, macrophages, mesenchymal stem cells, bone marrow-derived mast cells, as well as various nonhematopoietic cells. Its normal function is to regulate the balance between T-cell activation and tolerance through interaction with its two receptors: programmed death 1 (also known as PD-1 or CD279) and CD80 (also known as B7-1 or B7.1). PD-L1 is also expressed by tumors and acts at multiple sites to help tumors evade detection and elimination by the host immune system. PD-L1 is expressed in a broad range of cancers with a high frequency. In some cancers, expression of PD-L1 has been associated with reduced survival and unfavorable prognosis. Antibodies that block the interaction between PD-L1 and its receptors are able to relieve PD-L1-dependent immunosuppressive effects and enhance the cytotoxic activity of antitumor T cells in vitro.

### Anti-PD-L1 Antibodies

Antibodies that specifically bind and inhibit PD-L1 activity (e.g., binding to PD-1 and/or CD80) are useful for the treatment of lung cancer (e.g., non-small cell lung cancer). Virtually any anti-PD-L1 antibody known in the art can be used in the methods of the invention. Suitable anti-PD-L1 antibodies include, for example, known anti-PD-L1 antibodies, commercially available anti-PD-L1 antibodies, or anti-PD-L1 antibodies developed using methods well known in the art. Anti-PD-11 antibodies include, without limitation, MEDI4736, MPDL3280A (Genentech/Roche), BMS-936559 (Bristol Myers Squibb), and MSB0010718C (Merck Serono).

MEDI14736 is an exemplary anti-PD-L1 antibody that is selective for PD-L1 and blocks the binding of PD-L1 to the PD-1 and CD80 receptors. MEDI4736 can relieve PD-L-mediated suppression of human T-cell activation in vitro and inhibits tumor growth in a xenograft model via a T-cell dependent mechanism.

Information regarding MEDI4736 (or fragments thereof) for use in the methods provided herein can be found in U.S. Pat. No. 8,779,108/US 2013/0034559, the disclosures of which are incorporated herein by reference in their entirety. The fragment crystallizable (Fc) domain of MEDI4736 contains a triple mutation in the constant domain of the IgG 1 heavy chain that reduces binding to the complement component C1q and the Fcγ receptors responsible for mediating antibody-dependent cell-mediated cytotoxicity (ADCC).

MEDI4736 and antigen-binding fragments thereof for use in the methods provided herein comprises a heavy chain and a light chain or a heavy chain variable region and a light chain variable region. In a specific aspect, MEDI4736 or an antigen-binding fragment thereof for use in the methods provided herein comprises a light chain variable region comprising the amino acid sequence of SEQ ID NO: 1 and a heavy chain variable region comprising the amino acid sequence of SEQ ID NO:2. In a specific aspect, MEDI14736 or an antigen-binding fragment thereof for use in the methods provided herein comprises a heavy chain variable region and a light chain variable region, wherein the heavy chain variable region comprises the Kabat-defined CDR1, CDR2, and CDR3 sequences of SEQ ID NOs:3-5, and wherein the light chain variable region comprises the Kabat-defined CDR1, CDR2, and CDR3 sequences of SEQ ID NOs:6-8. Those of ordinary skill in the art would easily be able to identify Chothia-defined, Abm-defined or other CDR definitions known to those of ordinary skill in the art. In a specific aspect, MEDI4736 or an antigen-binding fragment thereof for use in the methods provided herein comprises the variable heavy chain and variable light chain CDR sequences of the 2.14H9OPT antibody as disclosed in U.S. Pat. No. 8,779,108/US 2013/0034559, the disclosures of which are incorporated herein by reference in their entirety.

### Epidermal Growth Factor Receptor (EGFR)

Epidermal growth factor receptor (EGFR, Erb 1 or HER1) is a transmembrane glycoprotein of 170 kDa that is encoded by the c-erbB 1 proto-oncogene. EGFR is a member of the human epidermal growth factor receptor (HER) family of receptor tyrosine kinases (RTK) which includes HER2 (ErbB2), HER3 (ErbB3) and HER4 (ErbB4).

Receptor tyrosine kinases are important in the transmission of biochemical signals which initiate cell replication. They are large enzymes which span the cell membrane and possess an extracellular binding domain for growth factors such as epidermal growth factor (EGF) and an intracellular portion which functions as a kinase to phosphorylate tyrosine amino acids in proteins and hence to influence cell proliferation. Various classes of receptor tyrosine kinases are known (Wilks, Advances in Cancer Research, 1993, 60, 43-73) based on families of growth factors which bind to different receptor tyrosine kinases. The classification includes Class I receptor tyrosine kinases comprising the EGF family of receptor tyrosine kinases such as the EGF, TGFα, NEU, erbB, Xmrk, HER and let23 receptors, Class II receptor tyrosine kinases comprising the insulin family of receptor tyrosine kinases such as the insulin, IGFI and insulin-related receptor (IRR) receptors and Class III receptor tyrosine kinases comprising the platelet-derived growth factor (PDGF) family of receptor tyrosine kinases such as the PDGFα, PDGFβ and colony-stimulating factor 1 (CSF1) receptors. It is known that Class I kinases such as the EGF family of receptor tyrosine kinases are frequently present in common human cancers such as breast cancer (Sainsbury et. al., Brit. J. Cancer, 1988, 58, 458; Guerin et al., Oncogene Res., 1988, 3, 21 and Klijn et al., Breast Cancer Res. Treat., 1994, 29, 73), non-small cell lung cancers (NSCLCs) including adenocarcinomas (Cerny et al., Brit. J. Cancer, 1986, 54, 265; Reubi et al., Int. J. Cancer, 1990, 45, 269; and Rusch et al., Cancer Research, 1993, 53, 2379) and squamous cell cancer of the lung (Hendler et al., Cancer Cells, 1989, 7, 347), bladder cancer (Neal et. al., Lancet, 1985, 366), oesophageal cancer (Mukaida et al., Cancer, 1991, 68, 142), gastrointestinal cancer such as colon, rectal or stomach cancer (Bolen et al., Oncogene Res., 1987, 1, 149), cancer of the prostate (Visakorpi et al., Histochem. J., 1992, 24, 481), leukaemia (Konaka et al., Cell, 1984, 37, 1035) and ovarian, bronchial or pancreatic cancer (European Patent Specification No. 0400586). It is also known that EGF type tyrosine kinase activity is rarely detected in normal cells whereas it is more frequently detectable in malignant cells (Hunter, Cell, 1987, 50, 823). It has been shown more recently (W. J. Gullick, Brit. Med. Bull., 1991, 47, 87) that EGF receptors which possess tyrosine kinase activity are overexpressed in many human cancers such as brain, lung squamous cell, bladder, gastric, breast, head and neck, esophageal, gynecological and thyroid tumors.

EGFR signaling is initiated by ligand binding followed by induction of conformational change, homodimerization or heterodimerization of the receptor with other ErbB family members, and trans-autophosphorylation of the receptor (Ferguson et al., Annu Rev Biophys, 37: 353-73, 2008), which initiates a signal transduction cascades that ultimately affects a wide variety of cellular functions, including cell proliferation and survival. Increases in expression or kinase activity of EGFR have been linked with a range of human cancers, making EGFR an attractive target for therapeutic intervention (Mendelsohn et al., Oncogene 19: 6550-6565, 2000; Grtlnwald et al., J Natl Cancer Inst 95: 851-67, 2003; Mendelsohn et al., Semin Oncol 33: 369-85, 2006). Increases in both the EGFR gene copy number and protein expression have been associated with favorable responses to the EGFR tyrosine kinase inhibitor, IRESSA® (gefitinib), in non-small cell lung cancer (Hirsch et al., Ann Oncol 18:752-60, 2007).

### EGFR Tyrosine Kinase Inhibitors (TKI)

Inhibitors of the tyrosine kinase enzyme in the epidermal growth factor receptor (EGFR) work by blocking the signals from the EGFR which lead to the growth and spread of tumors. Non-small cell lung cancer (NSCLC) characterized by epidermal growth factor receptor (EGFR) mutations have been shown to be sensitive to treatment with TKIs, as compared to those having wild-type EGFR. In various embodiments, the EGFR mutations activate EGFR signaling (e.g., via kinase activity) and/or occur in the EGFR kinase domain. Activating epidermal growth factor receptor (EGFR) mutations are found in four exons of the EGFR gene, exons 18 to 21, with around 90% of all mutations being the result of a deletion in exon 19 or an L858R point mutation in exon 21 (see e.g., Gazdar et al., Trends Mol Med 2004; 10: 481-486; Yoshida et al. J Thorac Oncol 2007; 2: 22-28; Forbes et al. Curr Protoc Hum Genet 2008; Chapter 10: Unit 10.11; Mok et al. N Engl J Med 2009; 361: 947-957; Wu et al. Clin Cancer Res 2011; 17: 3812-3821; Kim et al. Lung Cancer 2011; 71: 65-69; Kosaka ct al. Clin Cancer Res 2006; 12: 5764-5769; Masago et al. Jpn J Clin Oncol 2010; 40: 1105-1109; Mitsudomi et al. Lancet Oncology 2010; 11: 121-128; Sharma et al. Nature Rev Cancer 2007; 7: 169-181, the disclosures of each of which are incorporated herein by reference in their entirety). Without being bound to a particular theory, in mutated EGFR, tyrosine kinase inhibitors bind to the EGFR tyrosine kinase domain with high specificity and affinity, resulting in highly potent inhibition of aberrant signaling pathways. In some embodiments, this leads to significant tumor shrinkage in the majority of patients with EGFR mutation positive tumours.

First generation reversible tyrosine kinase inhibitors (TKIs) of EGFR include for example gefitinib (IRESSA®; AstraZeneca), erlotinib (Tarceva®; Genentech), and icotinib (BPI-2009H; Beta Pharma). Information regarding gefitinib for use in the methods provided herein can be found in U.S. Pat. No. 5,770,599, the disclosure of which is incorporated herein by reference in its entirety. Treatment with gefitinib (also known as IRESSA®) has been shown to result in tumor regression in some patients. However, resistance to first generation reversible tyrosine kinase inhibitors invariably develops after prolonged clinical use. In certain embodiments, EGFR having a threonine to methionine substitution at position 790 (T790M) is resistant to reversible tyrosine kinase inhibitors.

Second generation irreversible EGFR TKIs in late stage clinical development have the potential to overcome EGFR resistance to reversible tyrosine kinase inhibitors. Second generation irreversible EGFR TKIs include for example afatinib (Gilotrif®; BIRW 2992; Boehringer Ingelheim), dacomitinib (PF-00299804; Pfizer), and neratinib (IIKI-272; Puma Biotechnology). Second generation irreversible inhibitors also have activity against other ERBB family members.

Third generation irreversible EGFR TKIs are mutant-selective and were designed to target mutant EGFR over wild type EGFR. In contrast, first and second generation EGFR inhibitors were originally designed to target wild type EGFR. Third generation irreversible EGFR TKIs include for example Rociletinib (CO-1686; Clovis Oncology) and AZD9291 (AstraZeneca). AZD9291 has the following formula:

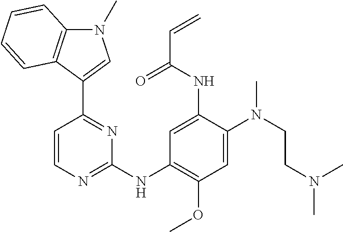 [SMILES: C=CC(=O)Nc1cc(Nc2nccc(-c3cn(C)c4ccccc34)n2)c(OC)cc1N(C)CCN(C)C]

AZD9291 (CAS no. 184475-35-2) is also known as N-(2-((2-(dimethylamino)ethyl)(methyl) amino)-4-methoxy-5-((4-(1-methyl-1H-indol-3-yl)pyrimidin-2-yl)amino)phenyl)acrylamide and 2-Propenamide, N-[2-[[2-(dimethylamino)ethyl]methylamino]-4-methoxy-5-[4-(1-methyl-1H-indol-3-yl)-2-pyrimidinyl]amino]phenyl]-). AZD9291 and uses thereof are described for example at US 2013/0053409, the disclosure of which is incorporated herein by reference in its entirety. In preclinical models, AZD9291 was effective against both EGFR-TKI sensitizing and resistance T790M mutations (Janne et al., J Clin Oncol 32:5s, 2014 suppl; abstr 8009).

### Selection of Anti-PD-L1 and EGFR TKI Treatment

Subjects suffering from lung cancer (e.g., non-small cell lung cancer) may be tested for EGFR mutations in the course of selecting a treatment method. Commercial tests for detecting EGFR mutations are available including for example, EGFR RGQ PCR Kit (Thermoscreen-QIAGEN), EGFR29 Mutation Detection (Amoy) PNAClamp EGFR Mutation Detection Kit (Panagene), Cobas® EGFR Mutation Test (Roche) and EGFR Pyro Kit (QIAGEN). Lab tests for detecting EGFR mutations have also been developed including for example the following techniques: PNA-LNA Clamp (Nagai et al. Cancer Res. 2005; 65: 7276-7282), Cycleave (Yatabe et al., J. Mol. Diagn. 2006; 8: 335-341), Invader (Naoki et al., Int. J. Clin. Oncol. 2011; 16: 335-344), Fragmcnt analysis for detecting delctions and inscrtions (Molina-Vila ct al., J. Thoracic. Oncol. 2008; 3: 1224-1235), and pyrosequencing (Dufort et al., J. Exp. Clin. Cancer Res. 2011; 30: 57). Patients identified as having tumors that are positive for EGFR activating mutations (e.g., mutations and deletions in the kinase domain) are identified as responsive to treatment with a combination of an anti-PD-L antibody and an EGFR tyrosine kinase inhibitor. Such patients are administered an anti-PD-L1 antibody, such as MEDI4736, or an antigen-binding fragment thereof in combination with an EGFR tyrosine kinase inhibitor, such as gefitinib.

In certain aspects, an NSCLC patient presenting with a solid tumor is administered MEDI4736 or an antigen-binding fragment thereof and an EGFR tyrosine kinase inhibitor, such as gefitinib. In certain aspects, the solid tumor is a non-small cell lung cancer (NSCLC) that is one or more of a squamous cell carcinoma, adenocarcinoma, large cell carcinoma, adenosquamous carcinoma and sarcomatoid carcinoma.

The intervals between doses of MEDI4736 or an antigen-binding fragment thereof can be about every two weeks. The EGFR tyrosine kinase inhibitor or gefitinib is administered every day. In certain aspects, the patient is administered one or more doses of an EGFR tyrosinc kinase inhibitor or gefitinib at a dose of about 250 mg/day. In certain aspects, administration of the EGFR tyrosine kinase inhibitor or gefitinib according to the methods provided herein is through enteral or enteric administration. In certain aspects, administration of the EGFR tyrosine kinase inhibitor or gefitinib according to the methods provided herein is through oral administration. For example, the EGFR tyrosine kinase inhibitor or gefitinib is formulated in a composition for oral administration (e.g., a pill or tablet).

In certain aspects the patient is administered two or more doses of MEDI4736 or an antigen-binding fragment thereof wherein the dose is about 3 mg/kg. In certain aspects the patient is administered two or more doses of MEDI4736 or an antigen-binding fragment thereof wherein the dose is about 10 mg/kg. In some embodiments, the at least two doses are administered about two weeks apart.

In certain aspects the patient is administered at least three doses of MEDI4736 or an antigen-binding fragment thereof wherein the dose is about 3 mg/kg. In certain aspects the patient is administered at least three doses of MEDI4736 or an antigen-binding fragment thereof wherein the dose is about 4 mg/kg. In certain aspects the patient is administered at least three doses of MEDI4736 or an antigen-binding fragment thereof wherein the dose is about 5 mg/kg. In certain aspects the patient is administered at least three doses of MEDI4736 or an antigen-binding fragment thereof wherein the dose is about 6 mg/kg. In certain aspects the patient is administered at least three doses of MEDI4736 or an antigen-binding fragment thereof wherein the dose is about 7 mg/kg. In certain aspects the patient is administered at least three doses of MEDI4736 or an antigen-binding fragment thereof wherein the dose is about 8 mg/kg. In certain aspects the patient is administered at least three doses of MEDI4736 or an antigen-binding fragment thereof wherein the dose is about 9 mg/kg. In certain aspects the patient is administered at least three doses of MEDI4736 or an antigen-binding fragment thereof wherein the dose is about 10 mg/kg.

In certain aspects the patient is administered at least four doses of MEDI4736 or an antigen-binding fragment thereof wherein the dose is about 3 mg/kg. In certain aspects the patient is administered at least four doses of MEDI4736 or an antigen-binding fragment thereof wherein the dose is about 4 mg/kg. In certain aspects the patient is administered at least four doses of MEDI4736 or an antigen-binding fragment thereof wherein the dose is about 5 mg/kg. In certain aspects the patient is administered at least four doses of MEDI14736 or an antigen-binding fragment thereof wherein the dose is about 6 mg/kg. In certain aspects the patient is administered at least four doses of MEDI4736 or an antigen-binding fragment thereof wherein the dose is about 7 mg/kg. In certain aspects the patient is administered at least four doses of MEDI4736 or an antigen-binding fragment thereof wherein the dose is about 8 mg/kg. In certain aspects the patient is administered at least four doses of MEDI4736 or an antigen-binding fragment thereof wherein the dose is about 9 mg/kg. In certain aspects the patient is administered at least four doses of MEDI4736 or an antigen-binding fragment thereof wherein the dose is about 10 mg/kg.

In certain aspects, about 3 mg/kg of MEDI4736, or an antigen-binding fragment thereof, is administered to a patient about every two weeks. In certain aspects, about 4 mg/kg of MEDI4736, or an antigen-binding fragment thereof, is administered to a patient about every two weeks. In certain aspects, about 5 mg/kg of MEDI4736, or an antigen-binding fragment thereof, is administered to a patient about every two weeks. In certain aspects, about 6 mg/kg of MEDI4736, or an antigen-binding fragment thereof, is administered to a patient about every two weeks. In certain aspects, about 7 mg/kg of MEDI4736, or an antigen-binding fragment thereof, is administered to a patient about every two weeks. In certain aspects, about 8 mg/kg of MEDI4736, or an antigen-binding fragment thereof, is administered to a patient about every two weeks. In certain aspects, about 9 mg/kg of MEDI4736, or an antigen-binding fragment thereof, is administered to a patient about every two weeks. In certain aspects, about 10 mg/kg of MEDI4736, or an antigen-binding fragment thereof, is administered to a patient about every two weeks.

In certain aspects, administration of MEDI4736, or an antigen-binding fragment thereof, according to the methods provided herein is through parenteral administration. For example, MEDI4736 or an antigen-binding fragment thereof can be administered by intravenous infusion or by subcutaneous injection. In some embodiments, the administration is by intravenous infusion. In certain aspects, administration of MEDI4736 or an antigen-binding fragment thereof according to the methods provided herein is through parenteral administration. For example, MEDI4736 or an antigen-binding fragment thereof can be administered by intravenous infusion or by subcutaneous injection. In some embodiments, the administration is by intravenous infusion.

In certain aspects, about 3 mg/kg of MEDI14736, or an antigen-binding fragment thereof, is administered to a patient about every two weeks and about 250 mg EGFR tyrosine kinase inhibitor or gefitinib is administered daily over the same period. In certain aspects, about 4 mg/kg of MEDI4736, or an antigen-binding fragment thereof, is administered to a patient about every two weeks and about 250 mg EGFR tyrosine kinase inhibitor or gefitinib is administered daily over the same period. In certain aspects, about 5 mg/kg of MEDI4736, or an antigen-binding fragment thereof, is administered to a patient about every two weeks and about 250 mg EGFR tyrosine kinase inhibitor or gefitinib is administered daily over the same period. In certain aspects, about 6 mg/kg of MEDI4736, or an antigen-binding fragment thereof, is administered to a patient about every two weeks and about 250 mg EGFR tyrosine kinase inhibitor or gefitinib is administered daily over the same period. In certain aspects, about 7 mg/kg of MEDI4736, or an antigen-binding fragment thereof, is administered to a patient about every two weeks and about 250 mg EGFR tyrosine kinase inhibitor or gefitinib is administered daily over the same period. In certain aspects, about 8 mg/kg of MEDI4736, or an antigen-binding fragment thereof, is administered to a patient about every two weeks and about 250 mg EGFR tyrosine kinase inhibitor or gefitinib is administered daily over the same period. In certain aspects, about 9 mg/kg of MEDI4736, or an antigen-binding fragment thereof, is administered to a patient about every two weeks and about 250 mg EGFR tyrosine kinase inhibitor or gefitinib is administered daily over the same period. In certain aspects, about 10 mg/kg of MEDI4736, or an antigen-binding fragment thereof, is administered to a patient about every two weeks and about 250 mg EGFR tyrosine kinase inhibitor or gefitinib is administered daily over the same period.

In some embodiments, at least two doses of MEDI4736 or an antigen-binding fragment thereof and at least about 13 doses of EGFR tyrosine kinase inhibitor, such as gefitinib are administered to the patient. In some embodiments, at least three doses, at least four doses, at least five doses, at least six doses, at least seven doses, at least eight doses, at least nine doses, at least ten doses, or at least fifteen doses or more of MEDI4736 or an antigen-binding fragment thereof can be administered to the patient. In some embodiments, MEDI4736 or an antigen-binding fragment thereof is administered over a two-week treatment period, over a four-week treatment period, over a six-week treatment period, over an eight-week treatment period, over a twelve-week treatment period, over a twenty-four-week treatment period, or over a one-year or more treatment period. In some embodiments, an EGFR tyrosine kinase inhibitor (e.g., gefitinib) is administered daily over a four-week treatment period, over an cight-wcck treatment period, over a twelve-week treatment period, over a sixteen-week treatment period, over a twenty-week treatment period, over a twenty-four-week treatment period, over a thirty-six-week treatment period, over a forty-eight-week treatment period, or over a one-year or more treatment period.

The amount of MEDI4736 or an antigen-binding fragment thereof and the amount of EGFR tyrosine kinase inhibitor (e.g., gefitinib) to be administered to the patient will depend on various parameters such as the patient's age, weight, clinical assessment, tumor burden and/or other factors, including the judgment of the attending physician. In further aspects the patient is administered additional follow-on doses. Follow-on doses can be administered at various time intervals depending on the patient's age, weight, clinical assessment, tumor burden, and/or other factors, including the judgment of the attending physician.

The methods provided herein can decrease or retard tumor growth. In some aspects the reduction or retardation can be statistically significant. A reduction in tumor growth can be measured by comparison to the growth of patient's tumor at baseline, against an expected tumor growth, against an expected tumor growth based on a large patient population, or against the tumor growth of a control population.

In certain aspects, a tumor response is measured using the Immune-related Response Criteria (irRc). In certain aspects, a tumor response is measured using the Response Evaluation Criteria in Solid Tumors (RECIST).

In certain aspects, a tumor response is detectable at week 8. In certain aspects, a tumor response is detectable after administration of about three or four doses of MEDI4736, or antigen-binding fragment thereof, and about 28 doses of gefitinib.

In certain aspects, a patient achieves disease control (DC). Disease control can be a complete response (CR), partial response (PR), or stable disease (SD).

A “complete response” (CR) refers to the disappearance of all lesions, whether measurable or not, and no new lesions. Confirmation can be obtained using a repeat, consecutive assessment no less than four weeks from the date of first documentation. New, non-measurable lesions preclude CR.

A “partial response” (PR) refers to a decrease in tumor burden >30% relative to baseline. Confirmation can be obtained using a consecutive repeat assessment at least 4 weeks from the date of first documentation.

“Stable disease” (SD) indicates a decrease in tumor burden of 30% relative to baseline cannot be established and a 20% increase compared to nadir cannot be established.

In certain aspects, administration of MEDI4736 or an antigen-binding fragment thereof and EGFR tyrosine kinase inhibitor (e.g., gefitinib) can increase progression-free survival (PFS).

In certain aspects, administration of MEDI4736 or an antigen-binding fragment thereof and EGFR tyrosine kinase inhibitor (e.g., gefitinib) can increase overall survival (OS).

In some embodiments, the patient has previously received treatment with at least one chemotherapeutic agent. The chemotherapeutic agent can be one or more of, for example, and without limitation, Gefitinib, Vemurafenib, Erlotinib, Afatinib, Cetuximab, Carboplatin, Bevacizumab, Erlotinib, and/or Pemetrexed.

In some embodiments, the tumor is refractory or resistant to at least one chemotherapeutic agent. The tumor can be refractory or resistant to one or more of, for example, and without limitation, Gefitinib, Vemurafenib, Erlotinib, Afatinib, Cetuximab, Carboplatin, Bevacizumab, Erlotinib, and/or Pemetrexed.

Treatment of a patient with a solid lung cancer tumor using both MEDI14736 or an antigen-binding fragment thereof and EGFR tyrosine kinase inhibitor, such as gefitinib (i.e., co-therapy) as provided herein can result in an additive and/or synergistic effect. As used herein, the term “synergistic” refers to a combination of therapies (e.g., a combination of MEDI4736 or an antigen-binding fragment thereof and EGFR tyrosine kinase inhibitor, such as gefitinib) which is more effective than the additive effects of the single therapies.

A synergistic effect of a combination of therapies (e.g., a combination of a MEDI4736 or an antigen-binding fragment thereof and EGFR tyrosine kinase inhibitor, such as gefitinib) may permit the use of lower dosages of one or more of the therapeutic agents and/or less frequent administration of said therapeutic agents to a patient with a solid lung cancer tumor. The ability to utilize lower dosages of therapeutic agents and/or to administer said therapies less frequently reduces the toxicity associated with the administration of said therapies to a subject without reducing the efficacy of said therapies in the treatment of a solid lung cancer tumor. In addition, a synergistic effect can result in improved efficacy of therapeutic agents in the management, treatment, or amelioration of a solid lung cancer tumor. The synergistic effect of a combination of therapeutic agents can avoid or reduce adverse or unwanted side effects associated with the use of either single therapy.

In co-therapy, MEDI4736 or an antigen-binding fragment thereof can be optionally included in the same pharmaceutical composition as the EGFR tyrosine kinase inhibitor, such as gefitinib, or may be included in a separate pharmaceutical composition. In this latter case, the pharmaceutical composition comprising MEDI4736 or an antigen-binding fragment thereof is suitable for administration prior to, simultaneously with, or following administration of the pharmaceutical composition comprising EGFR tyrosine kinase inhibitor, such as gefitinib. In certain instances, the MEDI4736 or an antigen-binding fragment thereof is administered at overlapping times as the EGFR tyrosine kinase inhibitor, such as gefitinib, in a separate composition.

### Kits

The invention provides kits for treating non-small cell lung cancer comprising an anti-PD-L antibody, such as MEDI4736, or an antigen-binding fragment thereof and an EGFR tyrosine kinase inhibitor, such as gefitinib. In various embodiments, the kit includes a therapeutic composition comprising MEDI4736 in a unit dose of between about 3 m/kg and about 10 mg/kg and/or gefitinib in a unit dose of 250 mg.

In some embodiments, the kit comprises a sterile container which contains a therapeutic and/or diagnostic composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired, the kit further comprises instructions for administering the anti-PD-L1 antibody and gefitinib to a subject having non-small cell lung cancer. In particular embodiments, the instructions include at least one of the following: description of the therapeutic agent; dosage schedule and administration for treatment or prevention of non-small cell lung cancer or symptoms thereof; precautions; warnings; indications; counter-indications; over dosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry, immunohistochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: NSCLC Patients Having EGFR Mutations were Responsive to Treatment of MEDI4736 and Gefitinib

A phase I, open-label, multicenter study (NCTO02088112) was performed to evaluate the safety, tolerability and efficacy of treatment with MEDI4736 in combination with the EGFR tyrosine kinase inhibitor (TKI) gefitinib in patients with Non-Small Cell Lung Cancer (NSCLC).

In the escalation phase of the study, patients were selected having locally advanced or metastatic NSCIC that either failed to respond or relapsed following any line of standard treatment, were unable to tolerate, or were not eligible for standard treatment (from 5 centers in USA, Japan, and Korea; aged ≥18 years). Escalation phase patients received MEDI4736 every 2 weeks (start dose 3 mg/kg) and gefitinib 250 mg once-daily for >1 year to establish the maximum tolerated dose (MTD) of the combination. In the expansion phase, patients identified as EGFR TKI-naïve/sensitive, EGFR mutation-positive NSCLC received (at MTD) MEDI4736 every 2 weeks and gefitinib, with or without 4 weeks of prior gefitinib treatment. Primary endpoints of the study included safety and tolerability of the combination of MEDI4736 and gefitinib (including MTD). Secondary endpoints of the study included antitumor activity of the combination, including RECIST 1.1 response.

MEDI4736 administered in combination with gefitinib was generally well tolerated in NSCLC patients (MEDI47363 mg/kg: n=3; 10 mg/kg: n=7).

Patients with EGFR mutation-positive disease were among those responsive to treatment with a combination of MEDI4736 (3 mg/kg) and gefitinib (Table 1). One patient having NSCLC positive for the EGFR Exon 19 deletion that received MEDI4736 (3 mg/kg) and gefitinib (Pt 1) showed a −13.04% change in lesion diameter after 8 weeks. Another patient having NSCLC positive for the EGFR Exon 19 deletion that received MEDI4736 (10 mg/kg) and gefitinib (Pt 9) showed a −26.09% change in lesion diameter after 8 weeks and a −13.04% change in diameter after 24 weeks.

TABLE 1
Escalation phase NSCLC patients receiving
MEDI4736 (3 mg/kg) and Gefitinib

| | | | Change in sum of | | |
|---|---|---|---|---|---|
| | | Treatment, | target lesion | | |
| | | days | diameter baseline- | EGFR | |
| MEDI4736 | Pt | Ongoing | 8 weeks, % | mutation | TKI |
| 3 mg/kg | 1 | 53 | −13.04 | Exon 19 Del | 1^{st} line |
| | | | | T790M | |
| | 2 | 53 | — | — | — |
| | 3 | 48 | — | — | — |
| | 4 | 45 | +67.86 | WT | — |
| | 5 | 72 | 0 | T790M | Naïve |
| | 6 | 15 | — | — | — |
| | 7 | 60 | 0 | Exon 19 Del | 1^{st} line |
| 10 mg/kg | 8 | 178 | −20.59 (−11.76^{a}) | WT^{b} | Naïve |
| | 9 | 264 | −26.09 (−13.04^{a}) | Exon 19 Del | Naïve |
| | 10 | 100 | +19.74 | WT | Naïve |

Del, deletion; WT, wild-type
^{a}Week 24
^{b}KRAS mutation

Thus, treatment with MEDI4736 and gefitinib was generally well tolerated in NSLCLC patients. Additionally, disease control was achieved in patients having EGFR mutation-positive NSCIC.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method of treating non-small cell lung cancer (NSCLC) in a human patient comprising administering to the patient an anti-PD-L1 antibody, or antigen binding fragment thereof, at a dosage from about 3 mg/kg to about 10 mg/kg every 2 weeks and an Epidermal Growth Factor Receptor (EGFR) tyrosine kinase inhibitor at about 250 mg per day, thereby treating the NSCLC in the patient.

2. (canceled)

3. The method of claim 1, wherein the patient is identified as having a non-small cell lung cancer that is positive for an EGFR activating mutation.

4. (canceled)

5. The method of claim 1, wherein the anti-PD-L1 antibody has one or more of a heavy chain CDR1 comprising the amino acid sequence GFTFSRYWMS (SEQ ID NO: 3); heavy chain CDR2 comprising the amino acid sequence NIKQDGSEKYYVDSVKG (SEQ ID NO: 4); heavy chain CDR3 comprising the amino acid sequence EGGWFGELAFDY (SEQ ID NO: 5); light chain CDR1 comprising the amino acid sequence RASQRVSSSYLA (SEQ ID NO: 6); light chain CDR2 comprising the amino acid sequence DASSRAT (SEQ ID NO: 7); and light chain CDR3 comprising the amino acid sequence QQYGSLPWT (SEQ ID NO: 8).

6. The method of claim 1, wherein the anti-PD-L1 antibody has one or more of a light chain comprising the amino acid sequence:
(SEQ ID NO: 1)
EIVLTQSPGTLSLSPGERATLSCRASQRVSSSYLAWYQQKPGQAPRLLIY
DASSRATGIPDRFSGSGSGTDFTLTISRLEPEDFAVYYCQQYGSLPWTFG
QGTKVEIK
and a heavy chain comprising the amino acid sequence:
(SEQ ID NO: 2)
EVQLVESGGGLVQPGGSLRLSCAASGFTFSRYWMSWVRQAPGKGLEWVAN
IKQDGSEKYYVDSVKGRFTISRDNAKNSLYLQMNSLRAEDTAVYYCAREG
GWFGELAFDYWGQGTLVTVSS.

7. The method of claim 1, wherein the anti-PD-L1 antibody is selected from MEDI4736, MPDL3280A, BMS-936559, and MSB0010718C.

8. The method of claim 1, wherein the EGFR tyrosine kinase inhibitor is one or more of gefitinib, erlotinib, icotinib, afatinib, dacomitinib, neratinib, rociletinib, and AZD9291.

9. (canceled)

10. The method of claim 1, wherein the non-small cell lung cancer is selected from the group consisting of squamous cell carcinoma, adenocarcinoma, large cell carcinoma, adenosquamous carcinoma and sarcomatoid carcinoma.

11. The method of claim 1, wherein the anti-PD-L1 antibody is administered at 3 mg/kg every 2 weeks.

12. The method of claim 1, wherein the anti-PD-L1 antibody is administered at 10 mg/kg every 2 weeks.

13. The method of claim 1, wherein the anti-PD-L1 antibody and EGFR tyrosine kinase inhibitor are administered for 8 weeks, 12 weeks, 16 weeks, 20 weeks or more.

14. The method of claim 1, wherein the method stabilizes or decreases one or more of tumor diameter, tumor volume, tumor mass, and tumor burden.

15. The method of claim 3, wherein the EGFR activating mutation is in the EGFR kinase domain.

16. The method of claim 15, wherein the activating mutation is a deletion in the EGFR kinase domain.

17. The method of claim 16, wherein the deletion comprises amino acids at positions 746-750 (ELREA) (SEQ ID NO: 13) of an EGFR polypeptide.

18. The method of claim 16, wherein the deletion is in a region encoded by exon 19 of an EGFR nucleic acid molecule.

19. The method of claim 1, wherein the administration of the anti-PD-L1 antibody, or an antigen-binding fragment thereof, is by intravenous infusion.

20. The method of claim 1, wherein the administration of the EGFR tyrosine kinase inhibitor is by oral administration.

21. The method of claim 1, wherein the patient is identified as responsive to treatment with an EGFR tyrosine kinase inhibitor.

22. The method of claim 1, wherein the patient is undergoing or has undergone treatment with an EGFR tyrosine kinase inhibitor.

23. The method of claim 1, wherein the EGFR polypeptide comprises a methionine at position 790.

24. The method of claim 1, wherein the method increases overall survival as compared to the administration of EGFR tyrosine kinase inhibitor alone.

25. The method of claim 1, wherein the anti-PD-L1 antibody, or antigen binding fragment thereof, is administered before, during, or after administration of the EGFR tyrosine kinase inhibitor.

26. The method of claim 1, wherein the anti-PD-L1 antibody, or antigen binding fragment thereof, is administered concurrently with the EGFR tyrosine kinase inhibitor.


SEQUENCE LISTING (13 sequences)

SEQ ID NO: 1 - protein, 108 aa, Artificial Sequence
  source: /note=“Description of Artificial Sequence Synthetic polypeptide”
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 2 - protein, 121 aa, Artificial Sequence
  source: /note=“Description of Artificial Sequence Synthetic polypeptide”
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 3 - protein, 10 aa, Artificial Sequence
  source: /note=“Description of Artificial Sequence Synthetic peptide”
GFTFSRYWMS

SEQ ID NO: 4 - protein, 17 aa, Artificial Sequence
  source: /note=“Description of Artificial Sequence Synthetic peptide”
NIKQDGSEKYYVDSVKG

SEQ ID NO: 5 - protein, 12 aa, Artificial Sequence
  source: /note=“Description of Artificial Sequence Synthetic peptide”
EGGWFGELAFDY

SEQ ID NO: 6 - protein, 12 aa, Artificial Sequence
  source: /note=“Description of Artificial Sequence Synthetic peptide”
RASQRVSSSYLA

SEQ ID NO: 7 - protein, 7 aa, Artificial Sequence
  source: /note=“Description of Artificial Sequence Synthetic peptide”
DASSRAT

SEQ ID NO: 8 - protein, 9 aa, Artificial Sequence
  source: /note=“Description of Artificial Sequence Synthetic peptide”
QQYGSLPWT

SEQ ID NO: 9 - protein, 176 aa, Homo sapiens
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 10 - DNA, 3349 bp, Homo sapiens
ggcgcaacgctgagcagctggcgcgtcccgcgcggccccagttctgcgcagcttcccgaggctccgcaccagccgcgcttctgtccgcctgcagggcattccagaaagatgaggatatttgctgtctttatattcatgacctactggcatttgctgaacgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatcccagtggagaaagttaaaaaa

SEQ ID NO: 11 - protein, 1210 aa, Homo sapiens [sequence omitted]

SEQ ID NO: 12 - DNA, 5616 bp, Homo sapiens
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctaaaaaaaaaaaaaaaaa

SEQ ID NO: 13 - protein, 5 aa, Homo sapiens
ELREA